(19) United States
(12) Patent Application Publication
(10) Pub. No.: US 2025/0107987 A1
(43) Pub. Date: Apr. 3, 2025

(54) NAIL POLISH COMPOSITION CONTAINING PIPERIDINOL COMPOUND

(71) Applicant: L'OREAL, Paris (FR)

(72) Inventors: Sam MORRISON, Piscataway, NY (US); Ramakrishnan HARIHARAN, Springfield, NJ (US); I-Fan HSIEH, Scotch Plaines, NJ (US)

(21) Appl. No.: 18/478,091

(22) Filed: Sep. 29, 2023

(51) Int. Cl. A61K 8/49; A61Q 3/02

(52) U.S. Cl. CPC A61K 8/4926; A61Q 3/02; A61K 2800/43; A61K 2800/54

(57) ABSTRACT

The present invention relates to nail compositions comprising at least one solvent, at least one coloring agent, at least one film forming agent, and at least one piperidinol compound, as well as to methods of making and using such compositions.

### FILED OF THE INVENTION

The present invention relates to nail compositions comprising at least one solvent, at least one coloring agent, at least one film forming agent, and at least one piperidinol compound, as well as to methods of making and using such compositions.

### DISCUSSION OF BACKGROUND

Traditional nail polish products contain a large amount of cellulose-based polymers such as nitrocellulose, primarily because cellulose-based polymers provide good adhesion of the compositions to nails upon application. Although nitrocellulose is the preferred film forming polymer for use in nail polish compositions and it constitutes the “gold standard” of film forming polymers in nail polish compositions, traditional nail compositions containing nitrocellulose are generally known for poor long wear characteristics and color instability when exposed to light and/or heat. The use of low levels of nitrocellulose tends to result in the coated films being easily damaged. On the other hand, the use of high levels of nitrocellulose results in the nail polishes being too hard and inflexible. Further, nitrocellulose does not result in a composition which is particularly color stable against light and/or heat.

In the past, proposed solutions to improve adhesion and gloss in nail compositions included incorporating into nitrocellulose-containing nail compositions high levels of plasticizers and replacing nitrocellulose with other alternative materials. For instance, U.S. Pat. No. 6,939,551 relates to the use of nitrocellulose in the presence of butyl phthalimide isopropyl phthalimide, and U.S. Pat. No. 8,790,669 discloses the use of latex film formers instead of nitrocellulose.

Proposed solutions also included adding UV filters such as octocrylene or benzophenone to nail compositions to try to increase color stability of the compositions against light and/or heat.

According to its abstract, U.S. Pat. No. 8,454,940 relates to compositions containing stabilizing mixtures of (a) an effective stabilizing amount of at least one merocyanine derivative having an absorption maximum of 350 to 400 nm, (b) at least one UV filter selected from benzotriazole derivatives; and optionally (c) at least one excited state quencher; for the protection of body care and household products against the deleterious effects of light, heat and oxygen.

Finally, some colorants found in nail polish such as red pigments are not particularly color stable against light and/or heat as well, which can result in changes in color intensity and/or color shade of the composition after exposure to light and/or heat.

There remains a need in the art for improved nail compositions which possess good color stability against light and/or heat in addition to good wear properties, particularly compositions which contain little or no UV filters.

Accordingly, one aspect of the present invention is a nail composition which possesses good color stability against light and/or heat in addition to possessing good wear properties, and in particular a composition which contains little or no UV filters.

### SUMMARY OF THE INVENTION

The present invention relates to nail compositions comprising at least one solvent, at least one coloring agent, at least one film forming agent, and at least one piperidinol compound. Preferably, the compositions further comprise at least one plasticizing agent. Also preferably, the compositions contain little or no UV filters. Specifically, the compositions contain little or no avobenzone, octocrylene, benzophenones, benzotriazoles and/or merocyanines.

The present invention also relates to methods of treating, caring for, protecting, enhancing the appearance of, and/or making up nails comprising applying compositions of the present invention to nails in an amount sufficient to treat, care for, enhance the appearance of, and/or make up the nails.

The present invention also relates to methods of improving color intensity stability and/or color shade stability against light and/or heat of nail compositions comprising at least one solvent, at least one coloring agent, and at least one film forming agent, wherein the methods comprise adding at least one piperidinol compound to the nail compositions during formation of the nail compositions in an amount sufficient to improve color intensity stability and/or color shade stability of the nail compositions against light and/or heat. Preferably, the nail compositions further comprise at least one plasticizing agent. Also preferably, the nail compositions contain little or no UV filters. Specifically, the compositions contain little or no avobenzone, octocrylene, benzophenones, benzotriazoles and/or merocyanines.

The present invention also relates to methods of making nail compositions comprising combining at least one solvent, at least one coloring agent, at least one film forming agent, and at least one piperidinol compound during formation of the nail compositions. Preferably, at least one plasticizing agent is also added during formation of the nail compositions. Also preferably, little or no UV filters are added during formation of the nail compositions. Specifically, the compositions contain little or no avobenzone, octocrylene, benzophenones, benzotriazoles and/or merocyanines.

It is to be understood that both the foregoing general description and the following detailed description are exemplary and explanatory only, and are not restrictive of the invention.

### DETAILED DESCRIPTION OF THE INVENTION

In the following description of the invention and the claims appended hereto, it is to be understood that the terms used have their ordinary and accustomed meanings in the art, unless otherwise specified.

“About” as used herein means within 10% of the indicated number (e.g., “about 10%” means 9%-11% and “about 2%” means 1.8%-2.2%).

“A” or “an” as used herein means “at least one.”

“At least one” means one or more and thus includes individual components as well as mixtures/combinations.

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc.

“Film former”, “film-forming polymer” or “film-forming agent” as used herein means a polymer or resin that leaves a film on the substrate to which it is applied, for example, after a solvent accompanying the film former has evaporated, absorbed into and/or dissipated on the substrate.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 115° C.

“Non-volatile”, as used herein, means having a flash point of greater than about 115° C.

“Polymer” as used herein means a compound which is made up of at least two monomers.

“Free” or “substantially free” or “devoid of” as it is used herein means that while it is preferred that no amount of the specific component be present in the composition, it is possible to have very small amounts of it in the compositions of the invention provided that these amounts do not materially affect at least one, preferably most, of the advantageous properties of the compositions of the invention. Thus, for example, “free of oil” means that an effective amount (that is, more than trace amounts) of oil(s) is/are omitted from the composition (that is, about 0% by weight), “substantially free of oil” means that oil(s) is/are present in amounts not greater than 0.1% by weight, and “devoid of oil” means that oil(s) is/are present in amounts not greater than 0.25% by weight, based on the total weight of the composition. The same nomenclature applies for all other ingredients identified throughout the application and in this paragraph such as, for example, UV filters and/or surfactants (compositions of the invention which are “free of UV filters,” “substantially free of UV filters,” and “devoid of UV filters,” as well as “free of surfactants,” “substantially free of surfactants,” and “devoid of surfactants,” have meanings consistent with the discussion within this paragraph), even if not specifically discussed for each identified ingredient in the application. Discussed examples of the use of such language such as those in this paragraph are intended to be exemplary, not limiting.

“UV filters” as it is used herein means sunscreen active agents approved by a governmental regulatory agency such as the Food and Drug Administration (FDA) in the U.S. or the EU Commission in Europe and includes avobenzone, octocrylene, benzophenones, benzotriazoles and merocyanines.

“Anhydrous” as it is used herein means that compositions of the invention contain less than 3% water, meaning that the compositions can also contain less than 2% water, and less than 1% water, as well as being “free of water,” “substantially free of water,” and “devoid of water.”

“Nails” means natural nails (finger and/or toe nails), as well as synthetic additions such as false nails.

“Physiologically acceptable” means compatible with nails and having a pleasant color, odor and feel, and which does not cause any unacceptable discomfort (stinging or tautness) liable to discourage a consumer from using the composition.

“Makeup Result” as used herein, refers to compositions where color remains the same or substantially the same as at the time of application, as viewed by the naked eye, after an extended period of time. “Makeup Result” may be evaluated by evaluating long wear properties by any method known in the art for evaluating such properties. For example, long wear may be evaluated by a test involving the application of a composition to nails and evaluating the color of the composition after an extended period of time. For example, the color of a composition may be evaluated immediately following application to nails and these characteristics may then be re-evaluated and compared after a certain amount of time. Further, these characteristics may be evaluated with respect to other compositions, such as commercially available compositions.

“Natural” as in the phrase “natural compound” refers to any compound derived directly from a natural substance such as a plant without having undergone any chemical modification.

“Compound of natural origin” refers to any compound derived from a natural compound which has undergone one or more chemical modifications, for example by organic synthesis reaction, without the properties of the natural compound having been modified.

“Synthetic compound” refers to any compound which is not a natural compound or a compound of natural origin.

“Room temperature” means 25° C.

“Atmospheric pressure” means 760 mmHg, i.e. 105 pascals.

“Color intensity stability” as it is used herein means stability against color of the composition visibly fading (diminishing).

“Color shade stability” as it is used herein means stability against color of the composition visibly changing. For example, where a nail polish composition contains nitrocellulose, the composition can become more yellow, which can manifest itself in a different color (for example, if the nail polish composition is blue, yellowing might cause the composition to become more green).

“Sheer nail polish composition” as it is used herein means a nail polish composition having a contrast ratio of 40 or less. Preferably, the nail polish compositions of the present invention have a contrast ratio of 35 or less, preferably 30 or less, and preferably 25 or less.

“Contrast ratio” is a calculation used to determine if a sample is more translucent or opaque. To determine contrast ratio, a sample is measured over a light and dark background using a spectrophotometer, and two measurements are then compared. When the sample has a contrast ratio (dark/light) greater than 99.9%, it is completely opaque. A number less than 99.9% indicates that some light is passing through the sample, with more light passing through as the contrast ratio gets lower (for example, a contrast ratio of 50 means more light is passing through than a contrast ratio of 60).

Determination of a contrast ratio is known in the art. An example of how to determine contrast ratio is as follows.

Samples are drawn down on Laneta cards having a black portion and a white portion using a BYK Gardner film applicator. All samples are drawn down to the same thickness (for example, 300 microns). Using a spectrophotometer (for example, Data Color 600), a measurement is taken over the light (white) section of the card, and a measurement is taken over the dark (black) section of the card. The contrast ratio is the ratio of dark measurement/light measurement.

The compositions and methods of the present invention can comprise, consist of, or consist essentially of the essential elements and limitations of the invention described herein, as well as any additional or optional ingredients, components, or limitations described herein or otherwise useful. For example, the solvent system of the nail compositions of the invention can “consist essentially of” alkyl acetates, optionally including one or more C2-C8 monoalcohol(s) such as ethanol.

For purposes of the present invention, the “basic and novel property” associated with compositions, components and methods which “consist essentially of” identified ingredients or actions is “color intensity and shade stability against light.

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

All U.S. patents or patent applications disclosed herein are expressly incorporated by reference in their entirety.

### Nail Compositions

In accordance with the present invention, nail compositions comprising solvent, at least one coloring agent, at least one film forming agent, and at least one piperidinol compound are provided.

### Solvent

According to the present invention, nail compositions comprising at least one solvent are provided. Preferably, the at least one solvent is chosen from organic and inorganic solvents, which can be either volatile or non-volatile. Most preferably, the at least one solvent is at least one alkyl acetate.

For example, suitable solvents may particularly be chosen from:
alkyl acetates, in particular C1-C6 alkyl acetates such as ethyl acetate, propyl acetate, and butyl acetate,
liquid ketones at ambient temperature such as methylethylketone, methylisobutylketone, diisobutylketone, isophorone, cyclohexanone and acetone,
liquid alcohols at ambient temperature including monoalcohols such as ethanol, isopropanol, diacetone-alcohol, 2-butoxyethanol and cyclohexanol,
liquid glycols at ambient temperature such as ethyleneglycol, propyleneglycol, pentyleneglycol and glycerol,
liquid propyleneglycol ethers at ambient temperature such as propyleneglycol monomethylether, propyleneglycol monomethyl ether acetate and dipropyleneglycol mono-n-butylether,
short-chain esters (comprising in total from 3 to 8 carbon atoms), in particularly alkyl acetates, such as ethyl acetate, methyl acetate, propyl acetate, n-propyl acetate, iso-propyl acetate, n-butyl acetate, iso-butyl acetate, ter-butyl acetate and isopentyl acetate,
liquid alkanes at ambient temperature such as decane, heptane, dodecane and cyclohexane,
liquid aromatic hydrocarbons at ambient temperature such as toluene and xylene, liquid silicones at ambient temperature such as, for example,
dimethicones, and
mixtures thereof.

According to preferred embodiments, the at least one solvent includes at least one C2-C8 monoalcohol. Examples of suitable C2-C8 monoalchols include, but are not limited to, ethanol, propanol, butanol, isopropanol and isobutanol.

Preferably, the nail compositions of the present invention are anhydrous.

Preferably, the nail compositions of the present invention are free, substantially free, or devoid of water.

Preferably, the nail compositions of the present invention are free, substantially free, or devoid of oil.

Preferably, the nail compositions of the present invention are free, substantially free, or devoid of surfactant.

Preferably, the at least one solvent is present in the nail compositions of the present invention in an amount ranging from about 20% to about 80% by weight, preferably from about 30% to about 75% by weight, preferably from about 40% to about 75%, preferably from about 50% to about 75%, and preferably from about 55% to about 70% by weight with respect to the weight of the composition, including all ranges and subranges therebetween such as, for example, 25% to 65%, 45% to 67%, 33% to 50%, etc.

### Coloring Agent

According to the present invention, nail compositions comprising at least one solvent are provided. Preferably, the at least one coloring agent is chosen from the group consisting of soluble dyes, pigments, nacres and glitter.

The term “soluble dyes” should be understood to refer to organic, Inorganic or organometallic compounds, soluble in the composition according to the invention and intended to color said composition.

Suitable dyes are, for example, Sudan Red, DC Red 17, DC Green 6, B-carotene, soybean oil, Sudan brown, DC Yellow 11, DC Violet 2, DC Orange 5 and Quinoline Yellow.

The term “nacres” should be understood to refer to iridescent particles of any shape, particularly produced by some mollusks in their shell or by synthetic means.

The term “pigments” should be understood to refer to inorganic or organic, white or colored particles of any shape, insoluble in the composition according to the invention and intended to color said composition.

The pigments may be white or colored, inorganic and/or organic. Of the inorganic pigments, mention may be made of titanium dioxide, optionally surface-treated, zirconium or cerium oxides, along with zinc, iron (black, yellow or red) or chromium oxides, manganese violet, ultramarine blue, chromium hydrate and iron blue, metallic powders such as aluminum powder, copper powder.

Of the organic pigments, mention may be made of carbon black, D & C type pigments, and lacquers based on cochineal carmine, barium, strontium, calcium, aluminum.

Mention may also be made of effect pigments such as particles comprising a natural or synthetic organic or inorganic substrate, for example glass, acrylic resins, polyester, polyurethane, polyethylene terephthalate, ceramics, aluminas and optionally coated with metallic substances such as aluminum, gold, copper, bronze, or with metal oxides such as titanium dioxide, iron oxide, chromium oxide, inorganic or organic pigments and mixtures thereof.

The pearlescent pigments may be chosen from white pearlescent pigments such as mica coated with titanium, or bismuth oxychloride, colored pearlescent pigments such as titanium mica coated with iron oxides, titanium mica coated with iron blue and chromium oxide in particular, titanium mica coated with an organic pigments of the aforementioned type and pearlescent pigments based on bismuth oxychloride.

Pigments with goniochromatic properties may be used, particularly liquid crystal or multilayer pigments.

Optical brighteners or fibers optionally coated with optical brighteners may also be used.

Preferably, the at least one coloring agent is present in the nail compositions of the present invention in an amount ranging from about 0.01% to about 50% by weight, preferably from about 0.1% to about 40% by weight, preferably from about 0.5% to about 25%, preferably from about 1% to about 10%, and preferably from about 1% to about 5% by weight with respect to the weight of the composition, including all ranges and subranges therebetween such as, for example, 2% to 15%, 25% to 50%, 3% to 8%, etc.

### Film Forming Agent

According to the present invention, nail compositions comprising at least one film forming agent are provided. Preferably, the at least one film forming agent is chosen from polymers of natural origin, acrylic polymers (homopolymers or copolymers), epoxy resins, polyester resins, and mixtures thereof.

Preferably, the at least one film forming agent is chosen from polysaccharide derivatives, such as cellulose or guar gum derivatives. Preferred polysaccharide derivatives include nitrocellulose or a polysaccharide ester or alkylether. The term “polysaccharide ester or alkylether” refers to a polysaccharide consisting of repeat units comprising at least two identical or different rings and having a degree of substitution per saccharide unit between 1.9 and 3, preferably between 2.2 and 2.9, and more particularly between 2.4 and 2.8. The term substitution refers to the functionalization of hydroxyl groups into ester and/or alkylether functions, and/or the functionalization of carboxyl groups into ester functions. In other words, it may consist of a polysaccharide, partially or totally substituted with ester and/or alkylether groups. Preferably, the hydroxyl groups may be substituted with C₂-C₄ ester and/or alkylether functions.

Particular mention may be made of cellulose esters (such as cellulose acetobutyrates or cellulose acetopropionates), cellulose alkylethers (such as ethylcelluloses), and ethylguars.

Suitable film forming agents of natural origin also include plant resins such as dammars, elemi, copals, benzoin; gums such as shellac, sandarac and mastic.

Suitable acrylic polymers include polymers resulting from the copolymerization of monomers chosen from the esters and/or amides of acrylic acid and/or of methacrylic acid. As examples of monomers of ester type, mention may be made of methyl methacrylate, ethyl methacrylate, butyl methacrylate, isobutyl methacrylate, 2-ethylhexyl methacrylate and lauryl methacrylate. As examples of monomers of amide type, mention may be made of N-t-butylacrylamide and N-t-octylacrylamide.

Suitable acrylic polymers include polymers obtained by copolymerization of ethylenically unsaturated monomers containing hydrophilic groups, preferably of nonionic nature, such as hydroxyethyl acrylate, 2-hydroxypropyl acrylate, hydroxyethyl methacrylate and 2-hydroxypropyl methacrylate.

Suitable acrylic polymers also include styrene acrylate copolymers and acrylate silicone copolymers.

The at least one film-forming polymer may also be chosen from synthetic polymers such as polyurethanes, vinyl polymers, polyvinylbutyrals, alkyd resins, ketone/aldehyde resins, and resins from aldehyde condensation products, such as aryl sulfonamide formaldehyde resins such as toluene or sulfonamide formaldehyde resin.

Suitable epoxy resins include, for example those disclosed in U.S. Pat. No. 5,001,175, the entire contents of which is hereby incorporated by reference. Non-limiting examples of suitable epoxy resins include tosylamide epoxy resins, such as those sold by Estron Chemical under the tradename Polytex™, e.g., E-75, E-100, and NX-55, NX-3214). Other non-limiting examples of suitable epoxy resins include aryl-sulfonamide epoxy resins.

Suitable film forming agents also include polyester resins formed by reacting a polyhydric alcohol with a polybasic acid, e.g., phthalic acid, such as the commercial product sold by Unitex Chemical Corporation under the name UNIPLEX 670-P, which is a polyester resin obtained by reacting trimellitic acid, neopentyl glycol, and adipic acid. (Meth) acrylic resins according to the disclosure may include copolymers of methyl methacrylate with butyl acrylate, butyl methacrylate, isobutyl methacrylate, or isobornyl methacrylate, for example, the commercial products PARALOID DM-55, PARALOID B48N, PARALOID B66, and ELVACITE 2550; copolymers of isobutylmethacrylate and butyl methacrylate, for instance, the commercial product ELVACITE 2046; and isobutyl methacrylate polymers, for example, PARALOID B67.

Specific examples of suitable film forming agents include, but are not limited to phthalic anhydride/glycerin/glycidyl decanoate copolymer, adipic acid/neopentyl glycol/trimellitic anhydride copolymer, tosylamide/epoxy resin, butyl acetate (and) acrylates copolymer, and hydrogenated acetophenone/oxymethylene copolymer.

Preferably, the at least one film forming agent is present in the nail compositions of the present invention in an amount ranging from about 1% to about 50% by weight, preferably from about 3% to about 40% by weight, preferably from about 5% to about 35%, preferably from about 7.5% to about 30%, and preferably from about 10% to about 25% by weight with respect to the weight of the composition, including all ranges and subranges therebetween such as, for example, 10% to 20%, 25% to 50%, 8% to 22%, etc.

### Piperidinol Compound

According to the present invention, nail compositions comprising at least one piperidinol compound are provided. The nail compositions of the present invention comprise a UV (ultraviolet) absorbing system comprising the at least one piperidinol compound. According to preferred embodiments, the UV absorbing system comprises at least 50% by weight of the at least one piperidinol, preferably at least 60% by weight of the at least one piperidinol, preferably at least 75% by weight of the at least one piperidinol, preferably at least 85% by weight of the at least one piperidinol, and preferably at least 90% by weight of the at least one piperidinol. According to one embodiment, the UV absorbing system contains only (consists of) the at least one piperidinol.

Piperidine is an organic compound with the molecular formula (CH₂)₅NH. It is a heterocyclic amine which is a six-membered ring containing five methylene bridges (—CH2-) and one amine bridge (—NH—).

Piperidinol is a piperidine compound which has at least one hydroxy (OH) substitution, and can contain up to 5 hydroxy substitutions such as, for example, 2 to 3 hydroxy substitutions, at carbon positions on the ring. Piperidinol can also be substituted with non-hydroxyl group substitutions at one or more carbon positions on the ring, if desired, and up to two such substitutions can occur at each ring position, if desired. Such additional non-hydroxyl group substitutions at one or more ring positions can include, but are not limited to, one or two substituted or unsubstituted, linear or branched, alkyl groups, preferably containing 1-8 carbon atoms, preferably 1-6 carbon atoms, preferably 1-3 carbon atoms, and preferably 1-2 carbon atoms.

Piperidinol can be in nonionic form, or in the form of a salt. Suitable salt forms of the piperidinol compound of the present invention include but are not limited to alkali metal salts (e.g., Na, K), alkaline earth salts (e.g., magnesium), and carboxylate salts such as salts of alpha hydroxy acids (e.g., lactates, citrates).

According to preferred embodiments of the present invention, the at least one piperidinol compound has one hydroxy substitution.

According to preferred embodiments, the at least one piperidinol compound has at least one methyl substitution, preferably two to six methyl substitutions, and preferably 4 methyl substitutions. According to preferred embodiments, the 4 methyl substitutions occur at two positions on the ring.

According to preferred embodiments, the at least one piperidinol compound is in the form of a salt. Preferably, the salt is a carboxylate salt, most preferably a citrate salt.

According to preferred embodiments of the present invention, the at least one piperidinol compound is tetramethylhydroxypiperidinol citrate. An example of a commercially-available product of this preferred piperidinol compound is Tinogard® Q available from BASF, which contains 10% active agent

Preferably, the at least one film piperidinol compound is present in the nail compositions of the present invention in an amount sufficient to inhibit yellowing of the composition and/or color-fading of the compositions as discussed above. Preferably, the at least one piperidinol compound is present in an amount ranging from about 0.015% to about 1.5% by weight, preferably from about 0.02% to about 1% by weight, preferably from about 0.02% to about 0.5%, preferably from about 0.02% to about 0.2%, and preferably from about 0.02% to about 0.1% by weight with respect to the weight of the composition, including all ranges and subranges therebetween such as, for example, 0.02% to 0.08%, 0.025% to 0.1%, 0.05% to 0.2%, etc.

### Additional Optional Ingredients

### Plasticizing Agent (Plasticizer)

According to preferred embodiments, nail compositions of the present invention may optionally further comprise at least one plasticizer. Generally speaking, plasticizers are additives used to optimize the mechanical properties of films. They tend to reduce the Glass Transition Temperature (Tg) and increase the softness and flexibility of the films.

Preferably, suitable plasticizers have a boiling point measured at ambient pressure of less than or equal to 285° C., preferably less than or equal to 270° C., and preferably less than or equal to 250° C. In the present specification, the boiling point values are to be considered accurate to ±2° C. owing to the uncertainties of boiling point measurement.

Any plasticizing agent typically found in nail polish compositions can be used. Examples of suitable plasticizers include, but are not limited to, glycols and their ester derivatives, esters of acids, in particular carboxylic acids, such as citrates, adipates, carbonates, tartrates, phosphates or sebacates, oxyethylenated derivatives, such as oxyethylenated oils, and their mixtures. For example, suitable plasticizing agents include, but are not limited to, diisobutyl adipate, the ester of tertbutyl acid and 2,2,4-trimethylpentane-1,3-diol, diethyl adipate, diethyl phthalate, dibutyl phthalate, dioctyl phthalate, butyl 2-ethylhexyl phthalate, dimethyl sebacate, dibutyl sebacate, ethyl stearate, 2-ethylhexyl palmitate, dipropylene glycol n-butyl ether, tributyl phosphate, tributoxyethyl phosphate, tricresyl phosphate, triphenyl phosphate, glycerol triacetate, butyl stearate, butyl glycolate, benzyl benzoate, butyl acetyltricinoleate, glyceryl acetyltricinoleate, dibutyl phthalate, diisobutyl phthalate, dioctyl phthalate, dimethoxyethyl phthalate, diamyl phthalate, triethyl citrate, tributyl citrate, tributyl acetylcitrate, tri (2-ethylhexyl) acetylcitrate, dibutyl tartrate, camphor, ethyl tosylamide and mixtures thereof.

Preferably, if present, the plasticizer is present in the nail compositions in an amount from about 0.1% to about 25% by weight, preferably from about 0.5% to about 20% by weight, preferably from about 1% to about 10% by weight, and preferably from about 3% to about 8% by weight, of the total weight of the composition, including all ranges and subranges there between.

### Thixotropic Agent

According to preferred embodiments, the nail compositions of the present invention may also optionally further include at least one thixotropic. The at least one thixotropic agent may be chosen, for example, from hydrophilic or organophilic clays, hydrophilic or hydrophobic fumed silicas, elastomeric organopolysiloxanes, and mixtures thereof.

Clays are silicates containing a cation that may be chosen from calcium, magnesium, aluminium, sodium, potassium, and lithium cations, and mixtures thereof. As used herein, the term “hydrophilic clay” means a clay that is capable of swelling in water; this clay swells in water and forms after hydration a colloidal dispersion.

Examples of such products include, but are not limited to, clays of the smectite family such as montmorillonites, hectorites, bentonites, beidellites, and saponites, clays of the vermiculite family, stevensite, and chlorites.

These clays may be of natural or synthetic origin.

Non-limiting examples of hydrophilic clays include smectites such as saponites, hectorites, montmorillonites, bentonites, beidellite and, in at least one embodiment, synthetic hectorites (also known as laponites), for instance, the products sold by the company Laporte under the names Laponite XLG, Laponite RD, and Laponite RDS (these products include, for example, sodium magnesium silicates and sodium lithium magnesium silicates); bentonites, for instance the product sold under the name Bentone HC by the company Rheox; magnesium aluminium silicates, which may be hydrated, for instance, the products sold by the company Vanderbilt Company under the names Veegum Ultra, Veegum HS, and Veegum DGT, and calcium silicates, such as the product in synthetic form sold by the company under the name Micro-cel C.

The organophilic clays are clays modified with chemical compounds that make the clay capable of swelling in solvent media.

The clay may be chosen, for example, from montmorillonite, bentonite, hectorite, attapulgite, sepiolite, and mixtures thereof. In one embodiment, the clay is chosen from bentonite and hectorite.

The chemical compound used to modify the organophilic clay may be chosen, for instance, from quaternary amines, tertiary amines, amine acetates, imidazolines, amine soaps, fatty sulfates, alkyl aryl sulfonates, amine oxides, and mixtures thereof.

Suitable organophilic clays include, but are not limited to, stearalkonium clays such as stearalkonium hectorite and stearalkonium bentonites such as those sold under the names Bentone 27V by the company Elementis, Tixogel LG by the company United Catalyst, and Claytone AF and Claytone APA by the company Southern Clay; quaternium-18 bentonites such as those sold under the names Bentone 3, Bentone 38, Bentone 27 V CG, and Bentone 38V by the company Elementis, Tixogel VP by the company United Catalyst, and Claytone 34, Claytone 40, and Claytone XL by the company Southern Clay; and quaternium-18/benzalkonium bentonites such as those sold under the names Claytone HT and Claytone PS by the company Southern Clay.

The hydrophilic fumed silicas may be obtained by high-temperature hydrolysis of a volatile silicon compound in an oxyhydric flame, producing a finely divided silica. Hydrophilic silicas have a large number of silanol groups at their surface. Such hydrophilic silicas are sold, for example, under the names Aerosil 130®, Aerosil 2000, Aerosil 2550, Aerosil 3000, and Aerosil 380® by the company Degussa, and Cab-O-Sil HS-5®, Cab-O-Sil EH-5®, Cab-O-Sil LM-1300, Cab-O-Sil MS-550, and Cab-O-Sil M-5® by the company Cabot.

The hydrophobic fumed silicas may be obtained by modification of the surface of the silica via a chemical reaction that generates a reduction in the number of silanol groups, these groups possibly being substituted, for example, with hydrophobic groups.

The hydrophobic groups may be chosen, for instance, from:
trimethylsiloxyl groups, which may be obtained by treating fumed silica in the presence of hexamethyldisilazane. Silicas thus treated are known as “Silica silylate” according to the CTFA (6th edition, 1995). They are sold, for example, under the references Aerosil R8120 by the company Degussa, and Cab-O-Sil TS-5300 by the company Cabot,
dimethylsilyloxyl or polydimethylsiloxane groups, which may be obtained by treating fumed silica in the presence of polydimethylsiloxane or dimethyldichlorosilane. Silicas thus treated are known as “Silica dimethyl silylate” according to the CTFA (6th edition, 1995). They are sold, for example, under the references Aerosil R9720 and Aerosil R9740 by the company Degussa, and Cab-O-Sil TS-610® and Cab-O-Sil TS-7200 by the company Cabot.

The at least one thixotropic agent, if present, may be present in the composition in an amount from 0.05 percent by weight, for example, preferably ranging from about 0.05 percent to about 15 percent by weight, preferably from about 0.5 percent to about 10 percent by weight, and preferably from about 0.75 percent to about 5 percent by weight, relative to the total weight of the composition, including all ranges and subranges therebetween such as, for example 1% to 3%, 0.5% to 7%, 5% to 15%, etc.

### Fillers

According to preferred embodiments, the nail compositions of the present invention may also optionally further include at least one filler. Suitable examples of fillers include mineral or organic particles of any shape, in sheet, spherical or oblong form, regardless of the crystallographic shape (for example sheet, cubic, hexagonal, orthorhombic, etc). Mention may be made of talc, mica, kaolin, polyamide (Nylon®) (Orgasol® from Atochem), poly-6-alanine and polyethylene powders, tetrafluoroethylene polymer powders (Teflon®), lauroyl-lysine, starch, boron nitride, acrylic acid copolymers (Polytrap® from Dow Corning) and silicone resin microbeads (Tospearls® from Toshiba, for example), elastomer polyorganosiloxane particles, precipitated calcium carbonate, magnesium carbonate and hydro-carbonate, hydroxyapatite, glass or ceramic microcapsules, metallic soaps derived from carboxylic organic acids having 8 to 22 carbon atoms, preferably from 12 to 18 carbon atoms, for example zinc, magnesium or lithium stearate, zinc laurate, magnesium myristate.

The at least one filler, if present, is preferably present in an amount ranging from about 0.01% to about 15% by weight, preferably from about 0.1% to about 5% by weight, preferably from about 0.5% to about 2.5%, in relation to the total weight of the nail compositions, including all ranges and subranges therebetween.

### Auxiliaries/Additives

According to preferred embodiments, the compositions of the present invention may also optionally further include at least one additive or auxiliary commonly used in cosmetic compositions and known to a person skilled in the art as being capable of being incorporated into said compositions. Such additives or auxiliaries may be chosen from preservatives, fragrances, oils, waxes, surfactants, antioxidants, agents for combating free radicals, spreading agents, wetting agents, dispersing agents, antifoaming agents, neutralizing agents, stabilizing agents, active principles chosen from essential oils, UV filters, moisturizing agents, vitamins, actives, proteins, ceramides, plant extracts, fibers, and the like, wetting agents and their mixtures. Although, preferably, nail compositions of the present invention are “free of,” “substantially free of,” or “devoid of” such additives.

A person skilled in the art will take care to select the optional additional additives and/or the amount thereof such that the advantageous properties of the composition according to the invention are not, or are not substantially, adversely affected by the envisaged addition.

Needless to say, the composition of the invention should be cosmetically or dermatologically acceptable, i.e., it should contain a non-toxic physiologically acceptable. The composition may be in any galenic form normally employed in the cosmetic and dermatological fields which is suitable for topical administration onto nails.

These auxiliary additives may be present in the composition in a proportion from 0% to 99% (such as from 0.01% to 90%) relative to the total weight of the composition and further such as from 0.1% to 50% (if present), including all ranges and subranges therebetween.

In accordance with the present invention, the nail compositions of the present Invention can be a basecoat (primer), a color coat or a topcoat (over coat). However, it should be understood that each coat in the nail composition, itself, can comprise one or more layers. Thus, for example, the at least one color coat can comprise one or more color coat layers; the at least topcoat can comprise one or more topcoat layers; the at least one basecoat can comprise one or more basecoat layers. Preferably, each basecoat, color coat and topcoat compositions contain three or fewer layers of compositions, preferably two or fewer layers of compositions, and preferably a single layer of compositions.

During application of the nail composition, the basecoat (if present) is typically applied directly to the nail, the color coat is typically applied either directly to the nail (if no basecoat is present) or to a previously-applied basecoat, and the topcoat (if present) is typically applied to a color coat.

According to preferred embodiments of the present invention, methods of treating, protecting, enhancing the appearance of, caring for and/or making up nails by applying compositions of the present invention to the nails in an amount sufficient to treat, enhance the appearance of, care for and/or make up the nails are provided. Preferably, “making up” the nails includes applying at least one coloring agent to the nails in an amount sufficient to provide color and/or optical effect to the nails.

In accordance with the preceding preferred embodiments, the compositions of the present invention are applied topically to the nails in an amount sufficient to treat, enhance the appearance of, care for and/or make up the nails. The compositions may be applied to the desired area as needed, preferably once or twice daily, more preferably once daily and then preferably allowed to dry before subjecting to contact such as with clothing or other objects (for example, clothes or a topcoat). Preferably, the composition is allowed to dry for about 1 minute or less, more preferably for about 45 seconds or less.

According to preferred embodiments, methods of improving color intensity stability and/or color shade stability against light and/or heat of nail compositions comprising at least one solvent, at least one coloring agent, and at least one film forming agent are provided, wherein the methods comprise adding at least one piperidinol compound to the nail compositions during formation of the nail compositions in an amount sufficient to improve color stability of the nail compositions against light and/or heat. Preferably, the nail compositions further comprise at least one plasticizing agent. Also preferably, the nail compositions contain little or no UV filters. Specifically, the compositions contain little or no avobenzone, octocrylene, benzophenones, benzotriazoles and/or merocyanines.

According to preferred embodiments, methods of making nail compositions comprising combining at least one solvent, at least one coloring agent, at least one film forming agent, and at least one piperidinol compound during formation of the nail compositions are provided. Preferably, at least one plasticizing agent is also added during formation of the nail compositions. Also preferably, little or no UV filters are added during formation of the nail compositions. Specifically, the compositions contain little or no avobenzone, octocrylene, benzophenones, benzotriazoles and/or merocyanines.

In accordance with the previous two embodiments, amounts of the at least one piperidinol compound sufficient to inhibit visible color intensity and/or color shade degradation of the composition are added to or combined with the nail compositions of the present invention (or ingredients thereof). Such effective amounts are sufficient to inhibit visible color intensity and/or color shade degradation under time and temperature conditions standard in the industry, such as for example after 24 hours in an Atlas CPS material testing weathering device.

The present invention also envisages kits and/or prepackaged materials suitable for consumer use containing one or more compositions according to the description herein, alone or in combination with makeup products such as basecoats, topcoats, removal compositions, etc. The packaging and application device for any subject of the invention may be chosen and manufactured by persons skilled in the art on the basis of their general knowledge, and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example 1—Sample Compositions

| | General Range | Specific Range |
|---|---|---|
| Ingredient | (wt %) | (wt %) |
| Piperidinol Compound | 0.015-1.5% | 0.02-0.2% |
| (tetramethylhydroxypiperidinol | | |
| citrate) | | |
| Solvent | 20-80% | 55-70% |
| Film Former | 1-50% | 10-25% |
| Colorant | 0.1-50% | 10-25% |
| Plascticizer | 0-25% | 3-8% |
| Thixotropic Agent/Filler | 0-30% | 1.25-7.5% |
| Other | QS | QS |

### Example 2—Color Stability Testing

Identical nail polish compositions were prepared, except invention composition 1 contained 0.2% Tinogard Q (0.02% active agent), comparative composition A contained 0.1% Tinogard Q (0.01% active agent), comparative composition B contained 0.34% benzophenone, and comparative composition C contained 0.34% octocrylene. The difference in concentrations of these identified ingredients was corrected by adding extra ethyl acetate solvent to all compositions (except comparative composition A) to reach 100% for each composition. Octocrylene and benzophenone are known color stabilizers in nail polish compositions, and testing conducted was to compare the ability of Tinogard Q to stabilize color of nail polish compositions to the ability of octocrylene and benzophenone to stabilize color in nail polish compositions.

Invention composition 1 and comparative compositions A-C were subjected to the following testing.

Initially, one set of all compositions was set aside as controls. A second set of compositions was subjected to sun testing using an Atlas CPS material testing weathering device for 24 hours.

Samples of the first and second sets of compositions were then drawn down on Laneta cards using a BYK Gardner film applicator. All samples were drawn down to the same thickness (300 microns). Then, L*a*b* values were determined for each sample using a Data Color 600 spectrophotometer,

For each composition, L′a*b* values for the set aside (control) sample was compared to the L′a*b* values for the sample subjected to sun testing to determine the difference in L′a*b* values, thereby determining the amount of fading for each composition resulting from the sun testing.

Invention composition 1 was found to have equivalent or better color stability properties than octocrylene and benzophenone (comparative compositions B and C). Comparative composition A containing 0.1% Tinogard Q possessed inferior color stability properties as compared to all other compositions tested.

Further testing was also conducted to determine color stability against heat. To determine such color stability, samples were held at 45° C. for one week and tested in the same manner as discussed above. These results surprisingly showed that only Invention composition A was heat stable, and that the other comparative compositions were not.

Finally, additional testing was conducted on known color stabilizers sold under the Tinogard® name for color stability against both heat and sun, specifically Tinoguard DA, Tinoguard TS, Tinoguard TL. Surprisingly, none of these compounds resulted in color stability of nail compositions against either heat or sun.

Tetramethylhydroxypiperidinol citrate (Tinogard Q) has been found to provide light and heat stability for nail polish in systems without ingredients such as benzophenone and octocrylene. Tinogard Q can be an excellent replacement for benzophenone and octocrylene for stabilizing color in nail polish compositions.

What is claimed is:

1. A nail composition comprising at least one solvent, at least one coloring agent, at least one film forming agent, and at least one piperidinol compound, wherein the composition is substantially free of avobenzone, octocrylene, benzophenones, benzotriazoles and/or merocyanines, preferably having a contrast ratio of 40 or less.

2. The nail composition of claim 1, further comprising at least one plasticizing agent, preferably tributyl citrate.

3. The nail composition of claim 1, wherein the at least one piperidinol compound is tetramethylhydroxypiperidinol citrate.

4. The nail composition of claim 1, wherein the at least one solvent is at least one alkyl acetate, preferably ethyl acetate, propyl acetate and/or butyl acetate.

5. The nail composition of claim 1, wherein the at least one solvent is at least one monoalcohol, preferably ethanol and/or isopropanol.

6. The nail composition of claim 1, wherein the at least one coloring agent is at least one pearlescent pigment.

7. The nail composition of claim 1, wherein the at least one film forming agent is selected from the group consisting of polymers of natural origin, acrylic polymers, epoxy resins, polyester resins, and mixtures thereof.

8. The nail composition of claim 1, wherein the at least one film forming agent is at least one polymer of natural origin, preferably at least one cellulose compound, preferably nitrocellulose.

9. The nail composition of claim 1, wherein the composition is a color coat, topcoat or basecoat.

10. A method of making up or protecting nails comprising applying the composition of any preceding claim to nails in an amount sufficient to make up or protect the nails.

11. A method of improving color intensity stability and/or color shade stability against light and/or heat of a nail composition comprising at least one solvent, at least one coloring agent, and at least one film forming agent, wherein the methods comprise: adding at least one piperidinol compound to the nail composition during formation of the nail composition in an amount sufficient to improve color intensity stability and/or color shade stability of the nail compositions against light and/or heat, preferably in an amount from about 0.02% to about 0.2% by weight with respect to the total weight of the composition.
